(19) United States
(12) Patent Application Publication
(10) Pub. No.: US 2018/0289383 A1
(43) Pub. Date: Oct. 11, 2018

(54) MEANS AND METHOD TO STOP BLEEDING FROM THE NOSE

(71) Applicant: Gel-e, Inc., College Park, MD (US)

(72) Inventor: Robert E. Fischell, Dayton, MD (US)

(21) Appl. No.: 15/482,013

(22) Filed: Apr. 7, 2017

(51) Int. Cl. A61B 17/24; A61M 31/00

(52) U.S. Cl. CPC A61B 17/24; A61M 31/00

(57) ABSTRACT

A device for stopping bleeding from inside a nostril of a human nose. The device being formed from a sponge-like material that has an upper portion having a size and shape adapted for insertion into the nostril of a human nose and the upper portion also being formed integral with a handle, wherein an outer surface of the upper portion being coated with a material that causes blood to clot.

### FIELD OF USE

This invention is in the field of methods and devices for the treatment of a nose that is bleeding.

### BACKGROUND OF THE INVENTION

There are many human patients who suffer severely from a nose that bleeds with bleeding that is difficult to control. That is particularly true for the many patients who are required to take blood thinners to prevent a stroke that can be caused by atrial fibrillations of the heart. In U.S. Pat. No. 8,932,560, M. B. Dowling et al describe the use of chitosan that is a natural polysaccharide that is modified with hydrophobic moieties that has the capability for being able to cause blood to clot. What is needed for patients who suffer from severe bleeding of the nose is a novel means and method to apply chitosan or other blood clotting composition to the interior surface within that nostril of the nose that is bleeding.

### SUMMARY OF THE INVENTION

The present invention is a means and method for controlling a severely bleeding nose, particularly for patients who are required to use a blood thinner to preclude the possibility of a stroke that can be a result of atrial fibrillation. The invention consists of a sponge-like material that is formed into the top section of a nasal insert that is slightly tapered so as to readily slide up into the bleeding nose and a bottom handle that is used for both inserting the bleed stopping device into the nose and removing it a few minutes later when the bleeding has been essentially stopped. The outside tapered surface of the bleed stopping device is coated and impregnated with the chitosan material that comes in contact with the inner surface of the nostril where the bleeding is occurring so as to cause the blood to clot to prevent further bleeding. The time for the bleeding to stop would typically be only a few minutes because of the ability of the chitosan to force a clotting of the blood at that point within the nostril where the bleeding is occurring.

Another novel feature of the present invention is a comparatively large diameter hole that extends for the entire length of the device which serves two important functions, namely: 1) to allow excess blood within the patient's nostril and nasal cavity to come out, and when that flow ceases, it allows the patient to breathe through that hole. Another important feature of this invention is that the device causes some mechanical pressure against the interior surface of the nose that is bleeding that also helps to stop that bleeding.

Thus one object of the present invention is to be able to insert a device coated with chitosan into the nostril of a patient's nose that is bleeding to form a blood clot in order to stop the bleeding.

Another object of this invention is to have the main body of the device be formed from a sponge-like material onto which and into which the chitosan is placed.

Still another object of this invention is to extend the sponge-like material downward from the portion that is inserted into the nose to form a handle that is held by the patient for both placing the device into the nostril that is bleeding and removing the device from the nose when the bleeding has been stopped.

Still another object of this invention is to have a hole that extends for the entire length of the device that allows the downward flow of any blood that has flowed above the top of the device and also allows the patient to breathe through the device until it is removed from the patient's nose.

Still another feature of this invention is to apply pressure against that interior portion of the nose that is bleeding to assist in stopping the bleeding.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a perspective view of the blood clotting device of the subject system;

FIG. 2 is a cross section of the blood clotting device for the treatment of a bleeding nose showing the coating of chitosan on the outer tapered surface of the upper portion of the device, the handle for placing and removing the device from the patient's nose and a hole extending through the center of the device for removal of blood and for breathing after the blood flow has ceased;

FIG. 3 is a top view of the blood clotting device shown in FIGS. 1 and 2; and,

FIG. 4 is a bottom view of the blood clotting device shown in FIGS. 1 and 2.

### DETAILED DESCRIPTION OF THE INVENTION

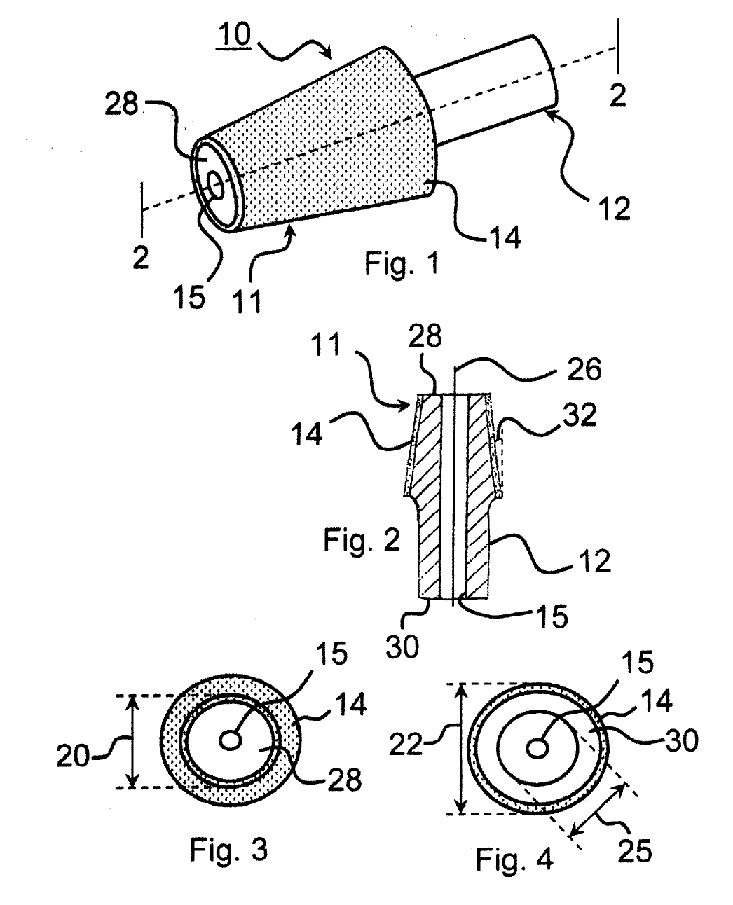

FIG. 1 is an angled view and FIG. 2 is a cross section of a nose-bleed stopping device 10 for insertion into a nostril of a patient's nose that has bleeding that is difficult to control. The device 10 consists of an upper portion 11 that is slightly tapered (or it could be cylindrical) that is formed as a single piece of sponge-like material with a handle 12, which handle is used as a convenient means for inserting the upper portion 11 of the device 10 into the nose and also for removing the device 10 from the nose after the bleeding from the nose has been stopped. The upper portion 11 has a coating of chitosan or and equivalent blood clotting agent that is able to cause blood to clot. The chitosan can be a coating of the upper portion 11 and it could also be contained for a short distance inside the outer surface of the sponge-like material from which the upper portion 11 is formed.

The bottom portion of the device 10 is a handle 12 that is formed as one piece with the upper portion 11 from a sponge-like material such as that which is used for ear plugs. The handle 12 is used for both inserting the upper portion 11 into the nostril and removing the device 10 from the nostril after the bleeding has been stopped by the chitosan coating of the upper portion 11 having been exposed to the opening in the nostril where from which the bleeding originates.

Another novel feature of the device 10 is a hole 15 that extends in the axial direction 2 through the entire length of the upper portion 11 and the handle 12. If there is free blood that has flowed into the nasal cavity prior to the insertion of the device 10, then this free blood can come out of the nose through the hole 15. Once there is no blood flowing through the hole 15, it can be used for the patient to breathe through that hole until the device 10 is removed from that person's nostril.

Referring to FIGS. 1-4, there is provided blood clotting device 10 for insertion into the nostril of a patient. As is seen in FIGS. 1 and 2, the blood clotting device 10 is formed of an upper portion 11 and a handle 12 adapted to be grasped by the patient for insertion and retraction of upper portion 11 into and out of the patient's nostril. In any event whether upper portion 11 and handle 12 are formed of a single composition or of different compositions, both sections are joined together to form a one piece tubular member. However, it is evident that handle 12 and upper portion 11 may be formed of different material compositions with the only limitation being the flexibility of upper portion 11. It is envisaged that handle 12 may be formed of either a flexible or rigid composition such as a plastic or rubber composition.

Blood clotting device 10 includes a through passage or hole 15 which extends throughout the length of the handle 12 and the upper portion 11 to provide an unrestricted flow path for passage of air and/or blood. As is seen in FIG. 2, the hole 15 extends in axial direction 26 to provide for the unrestricted flow path.

Blood clotting device upper portion 11 may be formed of an open cell plastic composition, a closed cell plastic composition, or composed of some type of porous material consisting of a fibrous skeleton formed from an invertebrate or other synthetic material. In a preferred embodiment, the handle 12 and the upper portion 11 are formed of a sponge composition with the important consideration being that the upper portion 11 defines a flexible upper portion 11. Handle 12 may be in the preferred embodiment formed of a sponge material of the same composition material as the upper portion 11 to provide a flexible overall insert into the patient's nostril. In some embodiments handle 12 may be formed of a hardened material such as a rigid plastic which is adhered to upper portion 11 by glue or other type of bond.

As is clearly seen in FIGS. 1 and 2, the upper portion 11 is tapered in axial direction 26 to allow ease of insertion into the patient's nostril. Upper portion 11 is generally formed in the shape of a truncated cone to permit insertion/retraction of the blood clotting device 10 without irritation to the patient.

Blood clotting composition 14 may be formed or comprised of a chitosan or some other type of blood clotting composition, for example, comprising one or more of fibrinogen and thrombin mixtures, thrombin, Factor XIII, Factor X, Factor VII (of human, animal, or synthetic origin), oxidized regenerated cellulose, cellulose, collagen, keratin, gelatin, chitosan, chitin, alginate, dextran, cyanoacrylate, PEG, albumin, glutaraldehyde, kaolin, bentonite, laponite, montmorillinite, galodinium, and aluminum chloride. In use, blood clotting composition 14 may be coated on an exterior surface of upper portion 11 and/or impregnated therein when upper portion 11 is formed of an open cell plastic or sponge composition. In all cases, blood clotting composition 14 forms a coating on upper portion 11 to provide contact with a bleeding blood vessel.

Again as seen in FIGS. 1-4, tapering of the upper portion 11 is seen in FIGS. 1 and 2, where upper portion 11 defines an upper surface 28 having an exterior diameter 20 which is less than the diameter 22 of lower surface 30 of upper portion 11.

As seen in FIG. 2, the upper portion 11 includes tapering angle 32 which in general has a tapering angle of less than 10 degrees with respect to axial direction 26. In cross-section, upper portion 11 takes the form of an annular contour defined by the outer surface of the blood clotting composition 14 and the through passage 15 as seen in FIGS. 1-4.

Handle 12 additionally defines an annular cross-section with respect to axial direction 26 and provides for an outer diameter 25 which is less than the upper portion lower surface 30 diameter 22. The dimensional relationships of the upper portion 11 and the handle member 12 are depicted in FIGS. 3 and 4 showing both top and bottom views of the blood clotting device 10.

To accommodate various sizes of noses that could have bleeding controlled by the device 10, it is expected that the device would come in different lengths and diameters to accommodate different sizes of a human nostril. The range of sizes would be from as large as 1.0 cm in diameter for the major diameter of the upper portion 11 or as small as 0.3 cm diameter to accommodate a very small nostril. The length of the upper portion 11 would be approximately 2 cm with a maximum possible length of 4 cm and a minimum possible length of 1.0 cm. The taper angle 32 of the upper portion 11 could be as little as zero degrees or as much as 10 degrees with an optimum taper being approximately 3 degrees. Although the handle 12 could have a great variety of lengths and diameters, an optimum diameter would be about 2 mm smaller than the diameter 22 at the bottom of the upper portion 11 and the length could be anything from ½ cm to as long as 5 cm. The hole 15 that extends through the entire length of the device 10 could have a diameter as small as 1.0 mm to as large as 7 mm with an ideal diameter being approximately 4 mm. It is contemplated that the upper portion 11 exerts an outward force on the inner surface of the bleeding nostril of the patient to promote the stopping of the bleeding.

In one step of a method for the treatment of a bleeding nostril of a human nose, a device from a sponge-like material that has an upper portion that is formed integral with a handle, the outer surface of the upper portion being coated with a material that causes blood to clot is provided to the person with a bleeding nostril. In a further step, the device is inserted into the nostril of the human nose where the bleeding originates by holding the handle with the thumb and at least one other finger. In a furthers step, the device is removed by pulling downward on the handle after the bleeding has been stopped.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A device for stopping bleeding from inside a nostril of a human nose, the device being formed from a sponge-like material that has an upper portion having a size and shape adapted for insertion into the nostril of a human nose and the upper portion also being formed integral with a handle, an outer surface of the upper portion being coated with a material that causes blood to clot.

2. The device of claim 1 wherein the material that causes the blood to clot is chitosan.

3. The device of claim 1 wherein the material that causes the blood to clot is situated both within the outer surface of upper portion of the device and also coats the outer surface of the upper portion of the device.

4. The device of claim 1 wherein the upper portion of the device is slightly tapered with the diameter being smaller at a top portion of the device and somewhat larger in diameter at a portion that connects to the handle.

5. The device of claim 1 further comprising a hole that extends through an entire length of the device.

6. A method for the treatment of a severely bleeding nostril of a human nose, the method consisting of:
a) forming a device from a sponge-like material that has an upper portion that is formed integral with a handle, the outer surface of the upper portion being coated with a material that causes blood to clot;
b) holding the handle with the thumb and one other finger;
c) inserting the device into the nostril of the human nose where the bleeding originates;
d) pulling downward on the handle to remove the device from the patient's nostril after the bleeding has been stopped.

7. The method of claim 6 wherein the material that causes the blood to clot is chitosan.

8. A blood clotting device for insertion into a nostril of a patient comprising:
(a) a substantially tubular member having a flexible upper portion and a handle, said flexible tubular member having a through passage extending in an axial direction; and,
(b) a blood clotting composition formed on the upper portion.

9. The device as recited in claim 8 where said upper portion is formed of an open or closed cell plastic composition.

10. The device as recited in claim 9 where said blood clotting composition is impregnated within said upper portion of the device.

12. The device as recited in claim 8 where said upper portion is formed of a porous material consisting of a fibrous skeleton formed from an invertebrate or a synthetic material.

13. The device as recited in claim 12 where said upper portion is a sponge composition.

14. The device as recited in claim 8 where said flexible upper portion and said handle are formed in one piece formation.

15. The device as recited in claim 8 where said upper portion is tapered in said axial direction.

16. The device as recited in claim 15 where said upper portion is contoured in the shape of a truncated cone for ease of insertion into and out of said nostril of said patient.

17. The device as recited in claim 18 where said upper portion defines an upper surface having an exterior diameter less than a diameter of a lower surface of said upper portion which is connected to said handle member in one piece formation.

18. The device as recited in claim 17 where said upper portion defines an annular cross-section when taken with respect to said axial direction.

19. The device as recited in claim 18 where said handle defines an annular cross-section with respect to said axial direction.

20. The device as recited in claim 19 where said handle includes an outer diameter less than an outer diameter of a lower surface of said upper portion.
